(19) United States
(12) Patent Application Publication
(10) Pub. No.: US 2026/0306837 A1
(43) Pub. Date: Oct. 8, 2026

(54) MICRO-CURRENT AND TEMPERATURE THERAPY SYSTEM AND DEVICE

(71) Applicant: Therabody, Inc., Los Angeles, CA (US)

(72) Inventors: Benjamin NAZARIAN, Beverly Hills, CA (US); Julia WEAVER, Los Angeles, CA (US); Jamie DACHISEN, Santa Monica, CA (US); Kevin XIE, Shenzhen, CA (US); Danny ZENG, Shenzhen (CN); Brian ZENG, Dongguan City (CN)

(21) Appl. No.: 19/638,434

(22) Filed: Apr. 3, 2026

Related U.S. Application Data
(60) Provisional application No. 63/783,622, filed on Apr. 4, 2025.

(51) Int. Cl. A61N 1/32; A61F 7/00; A61N 1/04

(52) U.S. Cl. CPC A61N 1/328; A61F 7/007; A61N 1/0472; A61F 2007/0052; A61F 2007/0075

(57) ABSTRACT

A treatment device for providing micro-current therapy and temperature therapy includes a main device generate electric current and a therapy module removably coupled to the main device to provide the micro-current therapy and the temperature therapy. The main device includes an air outlet and a fan. The therapy module includes a pair of applicators to be in contact with skin, a pair of controllable temperature elements to provide the temperature therapy, and an air inlet. The fan in the main device directs air flow from the air inlet to the air outlet.

### CROSS-REFERENCE TO RELATED APPLICATIONS

This application claims the benefit of U.S. Provisional Application No. 63/783,622, filed Apr. 4, 2025, which is incorporated herein by reference in its entirety.

### FIELD

The present disclosure relates to a temperature controlled micro-current system and device, more specifically, a treatment device that is configured to apply micro-current and temperature therapy, either independently or in combination with each other.

### BACKGROUND

Micro-current therapy devices apply low electric current to a skin surface to stimulate the muscles and the skin to improve appearance and achieve anti-aging effects. However, during application, micro-current therapy may temporarily cause discomfort and irritation, such that some users may be dissuaded from using the therapy as needed to achieve the desired effects.

The background description disclosed anywhere in this patent application includes information that may be useful in understanding the present disclosure. It is not an admission that any of the information provided herein is prior art or relevant to the presently claimed concepts, or that any publication specifically or implicitly referenced is prior art.

### SUMMARY

Some aspects of the present disclosure relate to a treatment device for providing micro-current therapy and temperature therapy. The treatment device includes a main device that generates electric current and a therapy module removably coupled to the main device to provide the micro-current therapy and the temperature therapy. The main device includes an air outlet and a fan. The therapy module includes a pair of applicators to be in contact with skin, a pair of controllable temperature elements to provide the temperature therapy, and an air inlet. The fan in the main device directs air flow from the air inlet to the air outlet.

In some aspects, the pair of applicators includes an anode electrode and a cathode electrode configured to provide the micro-current therapy while the applicators are in contact with the skin.

In some aspects, each applicator of the pair of applicators has an ovoid shape comprising a broader base tapering toward a narrower tip along a major axis.

In some aspects, a distance between the broader bases of the pair of applicators is in a range of 30 mm to 35 mm.

In some aspects, a distance between the narrower tips of the pair of applicators is in a range of 20 mm to 25 mm.

In some aspects, the therapy module further includes a housing having an upper cover, a bottom cover, and a sidewall between the upper cover and the bottom cover. The air inlet is disposed on the sidewall, and the pair of applicators is coupled to the upper cover.

In some aspects, each one of the pair of applicators has a maximum height as measured from the upper cover in a range of 13 mm to 22 mm.

In some aspects, the main device includes a handle portion and a head portion, and wherein the air outlet is disposed on the head portion.

In some aspects, the main device includes two air outlets disposed on opposite sides of the head portion.

In some aspects, an angle between the head portion and a vertical direction of the handle portion is in a range of 30 degrees to 80 degrees.

In some aspects, the treatment device is configured to administer the micro-current therapy and the temperature therapy simultaneously.

In some aspects, the micro-current therapy includes a current in a range of 100 microamps to 500 microamps.

In some aspects, the temperature therapy is a heating therapy providing a temperature in a range of 30° C and 50° C.

In some aspects, the temperature therapy is a cooling therapy providing a temperature below 20° C.

In some aspects, the therapy module further includes a heat sink to provide heat transfer to the controllable temperature elements.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIG. 1 is a perspective view of a temperature controlled micro-current therapy system according to some aspects.

FIG. 2 is a perspective exploded view of the temperature controlled micro-current therapy system according to some aspects.

FIG. 3 is a side exploded view of the temperature controlled micro-current therapy system according to some aspects.

FIG. 4 is a side sectional view of a main device of the temperature controlled micro-current therapy system according to some aspects.

FIG. 5 is a perspective exploded view of a therapy attachment of the temperature controlled micro-current therapy system according to some aspects.

FIG. 6 is a bottom perspective view of a housing of the therapy attachment of FIG. 5.

FIG. 7 is a top view of the therapy attachment of the temperature controlled micro-current therapy system according to some aspects.

FIG. 8 is a bottom view of the therapy attachment of the temperature controlled micro-current therapy system according to some aspects.

FIG. 9A and 9B are side views of the therapy attachment of the temperature controlled micro-current therapy system according to some aspects.

FIG. 10 is a front view of the therapy attachment of the temperature controlled micro-current therapy system according to some aspects.

FIG. 11 is a perspective view of a temperature controlled micro-current therapy system according to some aspects.

In the drawings, like reference numbers generally indicate identical or similar elements. Additionally, generally, the left-most digit(s) of a reference number identifies the drawing in which the reference number first appears.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description. References to one or more aspects in the present disclosure can be, but not necessarily are references to the same aspect; and, such references mean at least one of the aspects. If a component is not shown in a drawing then this provides support for a negative limitation in the claims stating that that component is “not” present. However, the above statement is not limiting and in another aspect, the missing component can be included in a claimed aspect.

Reference in this specification to “one aspect,” “an aspect,” “a preferred aspect” or any other phrase mentioning the word “aspect” means that a particular feature, structure, or characteristic described in connection with the aspect is included in at least one aspect of the-disclosure and also means that any particular feature, structure, or characteristic described in connection with one aspect can be included in any aspect or can be omitted or excluded from any aspect. The appearances of the phrase “in one aspect” in various places in the specification are not necessarily all referring to the same aspect, nor are separate or alternative aspects mutually exclusive of other aspects. Moreover, various features are described which may be exhibited by some aspects and not by others and may be omitted from any aspect. Furthermore, any particular feature, structure, or characteristic described herein may be optional. Similarly, various requirements are described which may be requirements for some aspects but not other aspects.

Where appropriate any of the features discussed herein in relation to one aspect or aspect of the disclosure may be applied to another aspect or aspect of the disclosure. Similarly, where appropriate any of the features discussed herein in relation to one aspect or aspect of the disclosure may be optional with respect to and/or omitted from that aspect or aspect of the disclosure or any other aspect or aspect of the disclosure discussed or disclosed herein.

The terms used in this specification generally have their ordinary meanings in the art, within the context of the disclosure, and in the specific context where each term is used.

Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted.

It will be appreciated that the same thing can be said in more than one way.

Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various aspects given in this specification.

Without intent to further limit the scope of the disclosure, examples of instruments, apparatus, methods and their related results according to the aspects of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long," “up,” “down,” “forward,” “inboard,” “outboard,” and “below” used herein are merely for ease of description and refer to the orientation of the components as shown in the figures. It should be understood that any orientation of the components described herein is within the scope of the present disclosure.

The terms “connected” or “coupled” and related terms are used in an operational sense and are not necessarily limited to a direct connection or coupling. The term "thermally coupled" means coupled in a way capable of conducting heat, and the term "thermally insulated" means separated by a substance that deters heat transfer.

The term “flexible” generally means bendable and adaptable under relatively little force. In the context of various aspects of the present disclosure, flexible is intended to describe the dynamic conforming nature of the personal temperature controlled device to the general shape of a portion of a person's body, such as wrist, ankle, neck, shoulder, back, chest, forehead, rib cage, arch, temple, palm, etc., directly or indirectly in contact with or otherwise engaging a surface of the personal temperature-controlled device. In addition, the term “approximately” is generally used to modify a numerical value above and below the set value by a variation of +/-10%.

Smaller and lighter therapy devices for providing micro-current and temperature therapies are desirable. One potential hurdle to a smaller form factor has been managing heat dissipation within a portable device to protect the internal components from overheating. Another potential hurdle is designing the geometry and angle of the therapy device such that the contacts are arranged to facilitate constant contact and easier maneuverability with the user’s skin during treatment. Accordingly, a need exists for a portable device that provides micro-current and temperature therapy while also providing efficient heat management and facilitating constant contact with the user’s skin as the device is maneuvered by the user.

Micro-current therapy applies low electric current to a skin surface to stimulate the muscles and the skin to improve appearance and achieve anti-aging effects. In particular, micro-current therapy can provide tightening, toning, firming, or contouring effects to skin to improve muscle tone and appearance. The electric current stimulation may build muscles, improve blood circulation, stimulate collagen production, and reduce inflammation. The micro-current therapy is less costly and less risky than a surgery, and micro-current therapy devices are available for use at home. However, at the same time, at home micro-current devices require more frequent use to achieve desired results.

Micro-current therapy devices require two electrical contact surfaces to be in contact with the skin surface in order to apply the low electric current. A handheld micro-current therapy device that is designed to be maneuvered across the skin surface manually by a user would benefit from electrical contacts having a geometry and orientation angles between the electrical contacts and the handheld portion of the therapy device to facilitate contact with the skin surface while the therapy device is moved across the skin surface.

Moreover, the application of micro-current therapy may temporarily cause discomfort and irritation as side effects. If some users are unable to tolerate the discomfort and irritation associated with the therapy, they are less likely to continue with the therapy, making it difficult to achieve the desired effect.

Temperature therapy, such as cooling and heating, may be applied to relieve the discomfort and irritation caused by a micro-current therapy. In particular, applying cooling therapy to skin may increase circulation, reduce puffiness, and improve skin tone. Various apparatuses have been devised to achieve the desired heat and/or cold transfer. For example, some users may use an ice pack at the treated area after the micro-current therapy, and some users may use a temperature controlled device for a more precise control of the temperature.

However, one issue with temperature controlled devices is that they are typically separate from micro-current therapy devices and therefore can only be used after the micro-current therapy. Additionally, applying temperature therapy after the micro-current therapy does not alleviate the potential discomfort while the micro-current therapy is applied. Therefore, a user needs to have multiple devices for a treatment, and the pain relief is not provided at the same time with the micro-current therapy. Another issue with temperature controlled devices is the lack of portability and maneuverability. Bulky devices are often used to heat and cool the affected areas to provide fast and efficient heat transfer. The size of these devices is typically necessary because components are not optimally arranged or configured to promote efficient operation. These heating and cooling devices are often heavy, need to be in a fixed position, and plugged into a continuous power supply. One potential hurdle to a smaller form factor has been managing heat dissipation within a portable device to protect the internal components from overheating.

Therefore, there is a need for a single form factor that is capable of applying both temperature therapy with the micro-current therapy, such that the temperature therapy can be applied during or after the micro-current therapy, without having to change devices. By combining temperature therapy and micro-current therapy in a single form factor with simultaneous delivery of both therapies, users may have an improved user experience for achieving skin toning benefits with a reduction of total treatment time. There is also a need for a light portable micro-current and temperature controlled device that provides heating and/or cooling therapy comparable to the bulky device while also providing efficient heat management. It is further desirable to provide a versatile multi-therapy device that can be used on different body parts of a user.

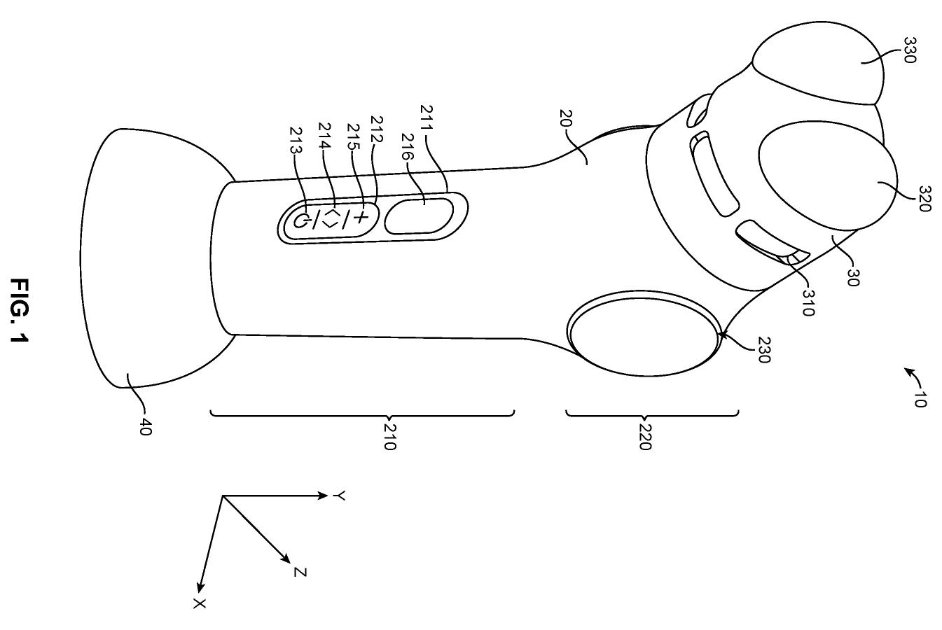

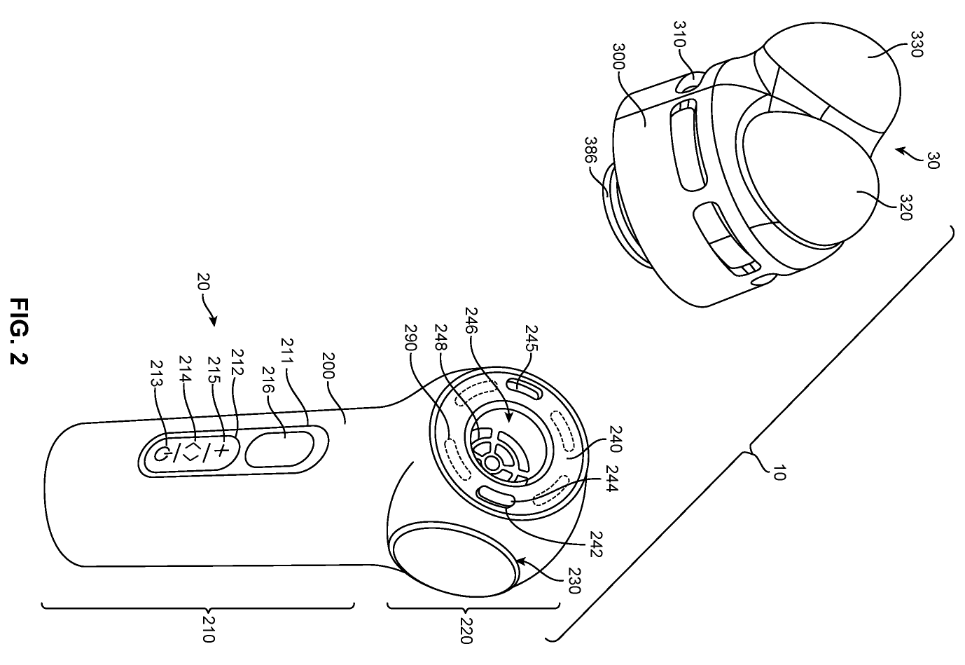

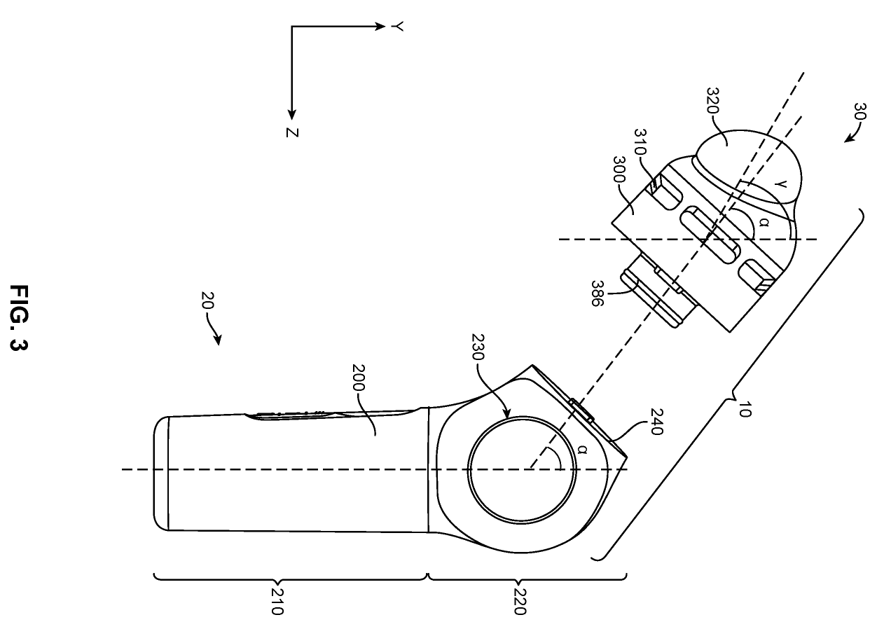

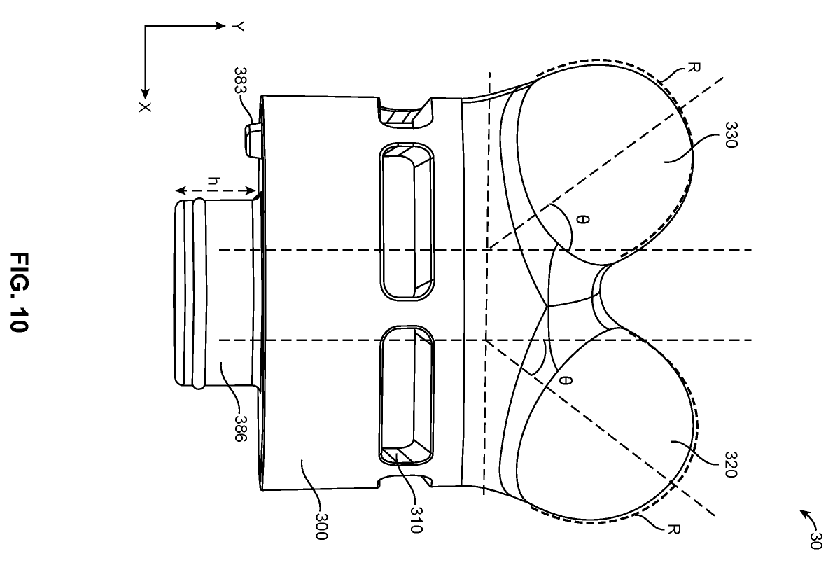

Described herein and shown in FIGS. 1–9 is an exemplary temperature controlled micro-current system that can provide the cooling and heating therapy during the micro-current therapy. FIG. 10 shows another temperature controlled micro-current according to a different aspect.

FIGS. 1–3 show a temperature controlled micro-current system 10 that can provide a micro-current therapy and a temperature therapy to a desired area on the user’s body and also promote air flow into and out of system 10. In some aspects, system 10 includes a main device 20 that can be held by the user and a therapy module 30 removably attached to main device 20. In some aspects, main device 20 and therapy module 30 when attached can provide the micro-current therapy and the temperature therapy, which can include cold therapy and/or hot therapy. In some aspects, the micro-current therapy and the temperature therapy are applied simultaneously during a treatment. In some aspects, the micro-current therapy and the temperature therapy are applied sequentially during a treatment. For example, the temperature therapy may be applied after the micro-current therapy is completed. In some aspects, the micro-current therapy and the temperature therapy are applied intermittently during a treatment. For example, the micro-current therapy may be applied for a predetermined time duration, such as 4–8 minutes, to treat a user’s face, followed by another predetermined time duration, such as 4–8 minutes of the temperature therapy. The predetermined time duration for each treatment may be preconfigured in a memory of the micro-current system 10 and/or set via user input with the micro-current system 10. In some aspects, the predetermined time duration for micro-current therapy may be the same as the predetermined time duration for temperature therapy. In some aspects, the predetermined time durations may vary. In another example, the micro-current therapy and temperature therapy may be applied sequentially to specific facial regions of the user for another predetermined time period, such as 1–2 minutes for each therapy. The predetermined time period or duration for each therapy may be based on one or more of recommended settings, preset settings, user-specific settings (e.g., one user may have higher tolerance for micro-current therapy and can set the predetermined time duration higher than another user). In aspects where the micro-current therapy and the temperature therapy are applied simultaneously, the total treatment time may be reduced as both therapies may work together to provide similar aesthetic benefits and skin tone improvements in one treatment session.

In some aspects, system 10 also includes a charging station 40. In some aspects, charging station 40 can be electrically coupled to a bottom of main device 20 for charging main device 20. In some aspects, charging station 40 and main device 20 can stand upwardly during charging.

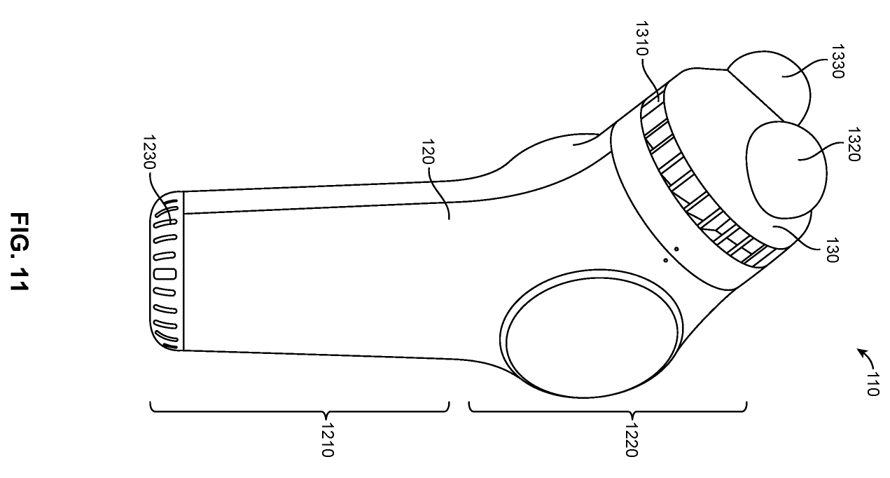

In some aspects, main device 20 and therapy module 30 are exclusively designed for each other, such that they only work when attached to each other. However, in some other aspects, main device 20 and therapy module 30 are not exclusively designed for each other. For example, in some aspects, main device 20 can alternatively connect with a second therapy module that provides other types of therapy, such as vibration therapy, LED light therapy, radiofrequency therapy, and/or massage therapy. In some aspects, therapy module 30 is compatible with another device to provide the same micro-current therapy and temperature therapy, for example, therapy module 30 can be removably attached to a main device 120 as shown in FIG. 11.

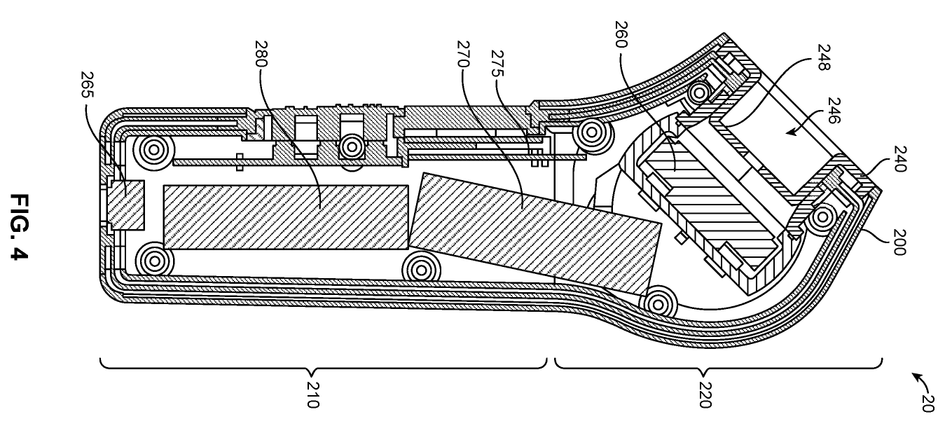

In some aspects, main device 20 has a housing 200 defining a handle portion 210 and a head portion 220. In some aspects, as shown in the sectional view of FIG. 4, housing 200 houses a fan 260, a charging port 265, a printed circuit board (PCB) 275, a battery 270, and a battery 280. In some aspects, fan 260 is arranged in the head portion 220 of main device 20. In some aspects, battery 270, battery 280, PCB 275, and charging port 265 are disposed within handle portion 210 of main device 20. In some aspects, battery 270 and battery 280 are rechargeable and may be charged through the charging port 265, for example, using a charging wire. While FIG. 4 illustrates two batteries 270 and 280 housed in main device 20, main device 20 may house a single battery or any number of batteries to power the system 10. In some aspects, PCB 275 in main device 20 may be configured to provide electrical and data communication with therapy module 30. In some aspects, PCB 275 may include a controller for various control functions for operating main device 20 and/or therapy module 30. In some aspects, PCB 275 may include a communication interface for wireless communication with external devices, such as a mobile device via, for example, a Bluetooth connection. In some aspects, operation of main device 20 and therapy module 30 may be controlled by a user through an application installed on a mobile device of the user by pairing the mobile device with the main device 20 and/or therapy module 30 through a Bluetooth connection.

In some aspects, fan 260 is configured to help dissipate heat generated in the therapy module 30 during operation and direct the heat as air flow out of the housing 200. In some aspects, main device 20 includes one or more air outlets 230 on an outer side of housing 200 to permit air to flow out of system 10 during temperature therapy, which will be described in further details below. In some aspects, main device 10 includes at least two air outlets 230 on opposite sides of housing 200 along a lateral direction (e.g. axis X). In some aspects, air outlets 230 are disposed on head portion 220. In some aspects, air outlets 230 are disposed away from charging port 265 of main device 20 to avoid interference with the charging function. For example, charging port 265 may be disposed on handle portion 210, so air outlets 230 on head portion 220 are opposite from the charging port 265. In some aspects, as shown in FIG. 11, air outlets 230 are disposed at a bottom of handle portion 210. In some aspects, air outlets 230 are configured such that they are exposed, partially or entirely, when a user is gripping on main device 20.

In some aspects, head portion 220 includes an attachment seat 240 defining the interface for connecting with therapy module 30 and potentially other types of modules. In some aspects, attachment seat 240 includes a center recess 246 and a securement protrusion 242 and a securement recess 243 outside of and along a perimeter of center recess 246. In some aspects, an air pathway 248 is defined within center recess 246 for air to flow from therapy module 30 to main device 20 and exit air outlet 230 during the temperature and/or micro-current treatment. In some aspects, center recess 246 is a cylindrical recess having a diameter in a range of 17 mm to 27 mm, such as 20 mm to 24 mm, such as 22 mm and a depth in a range of 12 mm to 22 mm, such as 15 mm to 19 mm, such as 17 mm. In some aspects, securement protrusion 242 includes electrical contacts 244 for electrically coupling with respective components on therapy module 30. In some aspects, securement recess 243 is configured for mechanically coupling with respective components on therapy module 30. In some aspects, electrical contacts 244 in securement protrusion 242 can be male electrical contacts. In some aspects, power is supplied from battery 270, 280, through electrical contacts 244 and to therapy module 30. In additional or alternative aspects, securement recess 243 may include male or female electrical contacts for electrically coupling with respective components on therapy module 30. It will be appreciated that the male and female electrical contacts in securement protrusion 242 and/or securement recess 243 can be reversed. In some aspects, head portion 220 includes magnets 290 for magnetically coupling with therapy module 30. In some aspects, magnets 290 are arranged within attachment seat 240 outside of and along the perimeter of center recess 246.

In some aspects, handle portion 210 extends along a vertical direction (e.g. axis Y), and head portion 220 is titled from the vertical direction and forming an angle α with handle portion 210. In some aspects, angle α is in a range of 30 degrees to 80 degrees, such as 40 degrees to 70 degrees, such as 60 degrees. In some aspects, angle α is designed to avoid blocking the user’s view during the treatment. In some aspects, therapy module 30 coupled to attachment seat 240 is also tilted at angle α relative to the vertical direction.

In some aspects, as shown in FIGS. 1–3, handle portion 210 has an approximately cylindrical shape that can be held by a single hand of the user. In some aspects, handle portion 210 has a different shape, such as a polygon prism. In some aspects, handle portion 210 has a user interface 211 comprising control buttons 212 and a display 216 for controlling the functions of main device 20 and therapy module 30. In some aspects, control buttons 212 and display 216 may be electrically coupled to the controller/PCB 275 in handle portion 210. In some aspects, control buttons 212 can control different modes, different intensities, different temperatures, etc. of the micro-current therapy and the temperature therapy. In some aspects, control buttons 212 can include one or more toggle buttons for toggling through different modes, different intensities, different temperature, etc. of the micro-current therapy and the temperature therapy and a power switch for turn on and off the power of main device 20. In some aspects, display 216 can show selections made through control buttons 212 and can also indicate a status of temperature controlled micro-current system 10, for example, whether the device is powered on, whether therapy module 30 (or another therapy module) is attached to main device 20, whether the device is charging, battery levels, Bluetooth connection status, treatment duration time, etc. In some aspects, control buttons 212 can include power button 213, modality button 214, and setting button 215. Long pressing power button 213 can power on or off main device 20 and therapy module 30. Modality button 214 can toggle between therapies available in therapy module 30 or in a second therapy module. In some aspects, main device 20 can connect with a second therapy module that provides other types of therapy, such as LED light therapy or heat and vibration therapy. In some aspects, short pressing modality button 214 can change which therapy is shown on display 216 and which therapy is available in the therapy module connected to the main device 20. In some aspects, short pressing modality button 214 can toggle between micro-current therapy, temperature therapy (e.g., heating therapy or cooling therapy), LED light therapy, and vibration therapy. Setting button 215 can adjust a setting of the selected therapy of the therapy module 30 or the second therapy module connected to main device 20. In some aspects, short pressing setting button 215 can change a setting, such as a microcurrent level and/or temperature level, of the therapy module 30 connected to main device 20. In some aspects, short pressing setting button 215 can change an LED light wavelength (e.g., red light, red and infrared light, yellow light, blue light, or the like), vibration intensity level (e.g., vibration motor frequency), and/or temperature setting of the second therapy module connected to main device 20.

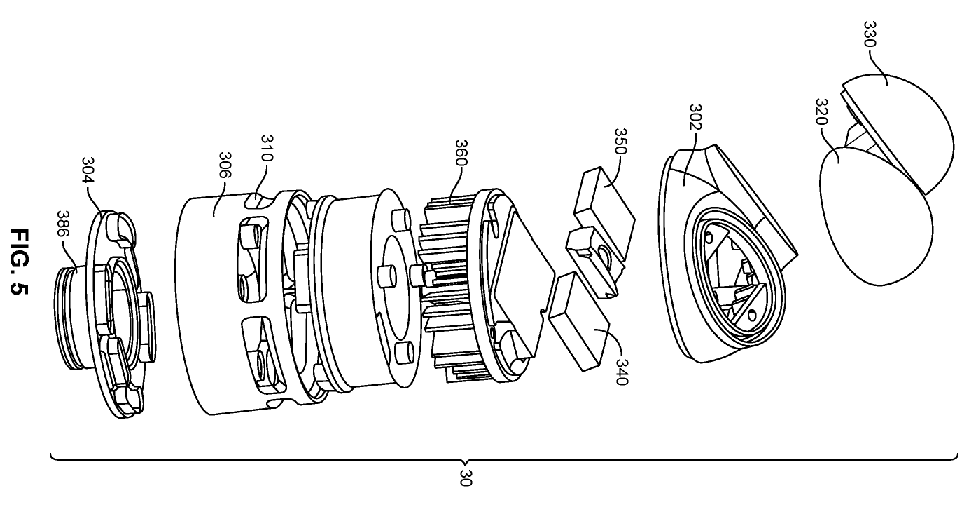

FIGS. 5–11 depict therapy module 30 according to some aspects. In some aspects, therapy module 30 includes a housing 300 having an upper cover 302, a bottom cover 304, and a sidewall 306. In some aspects, housing 300 houses controllable temperature elements 340, 350, and a heat sink 360. In some aspects, sidewall 306 has air inlet 310 to guide air flow into the housing 300. In some aspects, the air flow into air inlet 310 exits through air outlet 230 of main device 20. In some aspects, when therapy module 30 is coupled to main device 20, fan 260 in main device 20 helps pull the air flow from air inlet 310, through housing 300, into head portion 220 to exit through air outlet 230. In some aspects, a pair of applicators 320, 330 are disposed on upper cover 302. In some aspects, a bottom surface of bottom cover 304 defines an attachment surface 380 to be coupled with attachment seat 240 of main device 20.

In some aspects, when coupled, main device 20 and therapy module 30 form a fluid pathway via air inlet 310 through air pathway 248 and to air outlet 230 to facilitate heat dissipation within therapy module 30. Certain features of therapy module 30 may be based on forming the fluid pathway. In some aspects, therapy module 30 may be configured to selectively activate features based on the therapy module 30 being connected to a device having an air outlet to form a fluid pathway. For example, therapy module 30 may be configured to connect to a device that lacks an air outlet. In such a connection, therapy module 30 may selectively disable the temperature therapy feature.

In some aspects, applicators 320, 330 are removably coupled to upper cover 302. In some aspects, applicators 320, 330 are exposed and may be held in direct contact with the skin to apply the micro-current therapy and the temperature therapy. In some aspects, applicators 320, 330 can apply the micro-current therapy and the temperature therapy simultaneously during a treatment. In some aspects, applicators 320, 330 can apply the micro-current therapy and the temperature therapy sequentially during a treatment. For example, the temperature therapy may be applied after the micro-current therapy is completed. In some aspects, applicators 320, 330 can apply the micro-current therapy and the temperature therapy intermittently during a treatment.

In some aspects, applicator 320 and applicator 330 can simultaneously apply either the micro-current therapy or the temperature therapy. In some aspects, applicators 320 and 330 operate together to provide micro-current therapy, where one of applicators 320, 330 is an anode electrode, which serves as a positive electrode, and the other one of applicators 320, 330 is a cathode electrode, which serves as a negative electrode. In some aspects, applicator 320 and applicator 330 can apply the temperature therapy independently from each other. For example, in some aspects, only applicator 320 applies the temperature therapy during a treatment, while applicator 330 does not apply the temperature therapy.

The overall shape and geometry of applicators 320 and 330 are configured to maximize the surface area of applicators 320 and 330 to facilitate constant contact with the skin surface as the therapy module 30 is maneuvered across the skin surface. The shape and geometry of applicators 320 and 330 enable the contacts to maintain contact with the skin surface regardless of how handle portion 210 is held while also accommodating the additional circuitry needed to apply both micro-current and temperature therapy on the user’s skin.

In some aspects, applicators 320, 330 each have an ovoid shape or an egg shape.

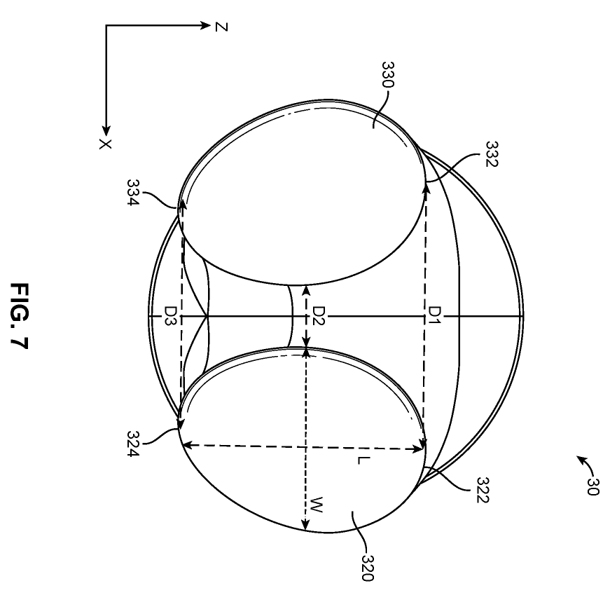

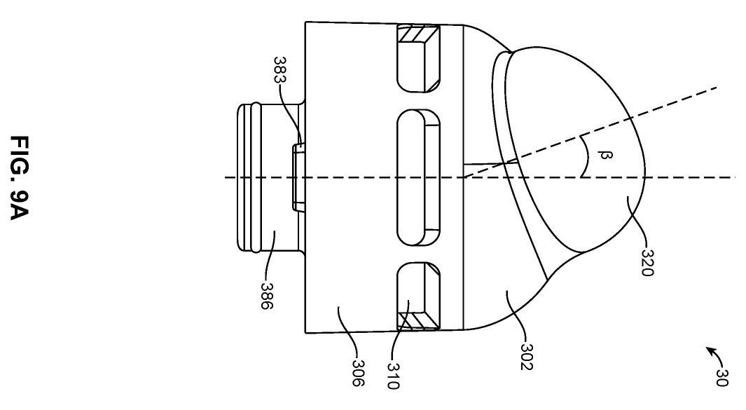

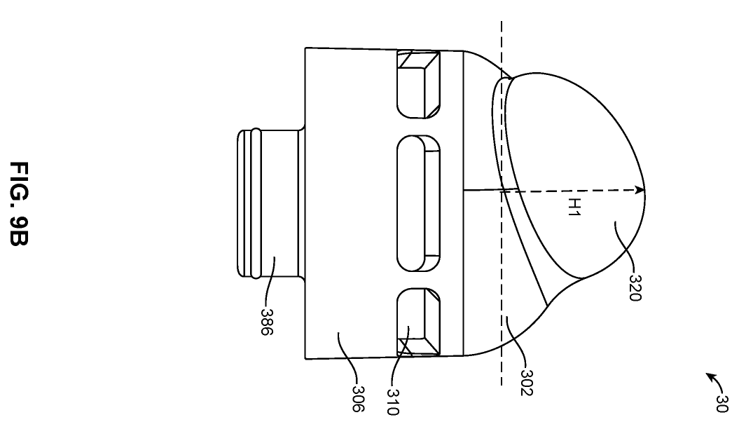

Applicators 320 has a broader base 322 that tapers to a narrower tip 324 along its major axis. Applicators 330 has a broader base 332 that tapers to a narrower tip 334 along its major axis. In some aspects, as shown in FIG. 7, each of applicators 320, 330 have a largest length L along axis Z in a range of 25 mm to 35 mm, such as 28 mm to 32 mm, such as 30 mm. In some aspects, each of applicators 320, 330 have a largest width W along axis X in a range of 16 mm to 26 mm, such as 19 mm to 23 mm, such as 21 mm. In some aspects, as shown in the side view of FIG. 9B, each of applicators 320, 330 have a maximum height H measured from upper cover 302 in a range of in 13 mm to 22 mm, such as 15 mm to 20 mm, such as 17 mm. In some aspects, as shown in the front view of FIG. 10, each of applicators 320, 330 have a radius of curvature R in a range of 10 mm to 20 mm, such as 13 mm to 17 mm, such as 15 mm.

In some aspects, applicators 320, 330 are distanced apart. As shown in FIG. 7, in some aspects, the narrowest distance d2 between applicators 320, 330 is in a range of 5 mm to 10 mm, such as 7 mm to 8 mm. In some aspects, the widest distance d1 between applicators 320, 330, as defined between broader bases 322, 332, is in a range of 30 mm to 35 mm, such as 32 mm to 33 mm. In some aspects, a distance d3 between narrower tips 324, 334 of applicators 320, 330 is in a range of 20 mm to 25 mm, such as 22 mm to 23 mm. In some aspects, applicators 320, 330 are tilted away from each other, such that they do not touch each other. For example, as shown in FIG. 11, the centerlines through applicators 320, 330 are titled away from the vertical direction (e.g. axis Y) on opposite sides and toward the lateral direction (e.g. axis X), forming an angle θ relative to the vertical direction (e.g. axis Y). In some aspects, angle θ is in a range of 30 degrees to 55 degrees, such as 35 degrees to 50 degrees, such as 45 degrees.

In some aspects, applicators 320, 330 are each oriented bending forward relative to housing 300, such that applicators 320, 330 are facing towards the user during the treatment. For example, as shown in FIG. 10, from a side view of therapy module 30, a centerline of applicator 330 is tilted away from the vertical direction (e.g. axis Y) and toward the longitudinal direction (e.g. axis Z) of housing 300, forming an angle β relative to the vertical direction (e.g. axis Y) of housing 300. In some aspects, angle β is in a range of 20 degrees to 50 degrees, such as 30 degrees to 40 degrees, such as 35 degrees. As discussed above, when therapy module 30 is attached to head portion 220 of main device 20, therapy module 30 is formed at angle α relative to the vertical direction (e.g. axis Y) due to the tilt of head portion 220. With angle β of applicators 320, 330 relative to housing 300, when therapy module 30 is attached to head portion 220 of main device 20, applicators 320, 330 are further titled away from the vertical direction (e.g. axis Y) at an angle γ, which is a sum of angle α and angle β. In some aspects, angle γ is in a range of 50 degrees to 100 degrees, such as 60 degrees to 90 degrees, such as 75 degrees.

In some aspects, the shape and the orientation of applicators 320, 330 are designed to improve contact with the skin and smoothness of the movement of applicators 320, 330 when gliding over the skin. It is understood that applicators 320, 330 can also have other applicable shapes. For example, in some aspects, applicators 320, 330 can have a spherical shape with a radius in a range of 10 mm to 20 mm, such as 13 mm to 17 mm, such as 15 mm.

In some aspects, therapy module 30 may have multiple components for providing the temperature therapy. In some aspects, therapy module 30 has one or more controllable temperature elements disposed within housing 300 and underneath the applicators. As shown in FIG. 5, in some aspects, therapy module 30 has two controllable temperature elements 340, 350 to correspond with each of applicator 320, 300. In some aspects, therapy module 30 has only one controllable temperature element. In some aspects, therapy module 30 has a heat sink 360 disposed within housing 300 and underneath controllable temperature elements 340, 350.

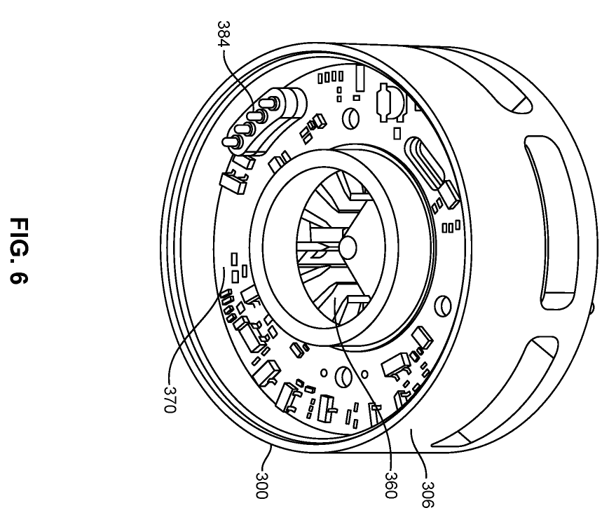

In some aspects, heat sink 360 may be disposed on lower surfaces of controllable temperature elements 340, 350, which can generate cooling or heating. In some aspects, upper surfaces of controllable temperature elements 340, 350 are approximate or in contact with applicators 320, 330, and the cooling or heating from heat sink 360 is transferred to applicators 320, 330 to provide cooling or heating therapy. Examples of controllable temperature elements 340, 350 include a Peltier device, Peltier heat pump, solid state refrigerator, thermoelectric cooler (TEC), etc. In some aspects, controllable temperature elements 340, 350 may adapt the Peltier effect to create a heat flux at the junction of two different types of materials, and transfer heat from one side of the device to the other. In some aspects, heat sink 360 can pull heat from the lower surfaces of applicators 320, 330, and when the therapy module 30 is coupled to main device 20, the fan 260 in main device 20 can help dissipate heat or direct heat away from the heat sink 360 and other components. In some aspects, controllable temperature elements 340, 350 can be coupled to a printed circuit board (PCB) 370 in housing 300 for electrical and data communication with main device 20, as shown in FIG. 6. In some aspects, the PCB 370 in housing 300 can further include a controller for various control functions of controllable temperature elements 340, 350 (e.g., turning on or off controllable temperature elements 340, 350, heating or cooling, etc.).

In some aspects, controllable temperature elements 340, 350 may be implemented as heat pumps that can directly convert electricity into heating and cooling power. In some aspects, when power is supplied to controllable temperature elements 340, 350, the current causes one side (cool side) of controllable temperature elements 340, 350 to absorb heat, and the opposite side of controllable temperature elements 340, 350 may then release heat (the hot side). For instance, when a user elects heating therapy from modality buttons 214 on main device 20, the upper sides of controllable temperature elements 340, 350 release heat, where the upper sides are in contact with the user. When a user elects cooling therapy from modality buttons 214 on main device 20, on the other hand, the upper sides of controllable temperature elements 340, 350 may absorb heat rather than releasing the heat to provide the cooling therapy to the user. Further, a user may elect cooling therapy and heating therapy to be applied alternately, and controllable temperature elements 340, 350 may be controlled to periodically alternate heat release and absorption functions. That is, controllable temperature elements 340, 350 may first cause heat to flow from one cool side to the hot side, and then current is reversed, controllable temperature elements 340, 350 may cause the heat to be moved in the opposite direction thereby reversing the hot side and the cold side. Consequently, the heating or cooling therapy can be selectively performed.

In some aspects, controllable temperature elements 340, 350, and heat sink 360 are configured to change the temperature of the therapy quickly, e.g., it may take about 2 seconds to drop 15° C at applicators 320, 330, or it may take about 1 second to increase 15° C at applicators 320, 330. In some aspects, fan 260 may be configured to run during and after a cooling treatment has been performed to help dissipate heat generated in therapy module 30 during operation. In some aspects, a micro-current and cooling therapy treatment may be performed for a first predetermined period of time (e.g., 10 minutes, 15 minutes, 20 minutes or the like), and the fan 260 may continue to run after the treatment for a second predetermined period of time (e.g., 2 minutes, 5 minutes, 10 minutes, or the like). In some aspects, the fan 260 may continue to run for a preset period of time after treatment, or the fan 260 may continue to run until the controllable temperature elements 340, 350 and/or heat sink 360 reaches a specific temperature level (e.g., room temperature ranging between 20 to 25 ° C). By continuing to run the fan 260 to dissipate heat after a treatment, the cooling performance of the controllable temperature elements 340, 350 and heat sink 360 may be optimized for subsequent treatments. For example, operating the fan 260 after a treatment may allow the applicators 320, 330 to cool down to room temperature (e.g., 20-25 ° C), which may allow the therapy module 30 to easily cool down for subsequent cooling therapy treatments of different users in a spa or clinical environment.

Based on the disclosure provided herein, one of ordinary skill in the art will recognize the various possible reconfigurations of controllable temperature elements that would achieve a heating/cooling effect.

In some aspects, controllable temperature elements 340, 350 may each have dimensions of approximately, e.g., 15 mm (L) × 15 mm (W) × 4 mm (D). In some aspects, a top surface of the heat sink 360 may a surface area of approximately, e.g., 20 mm (L) × 30 mm (W), and a bottom cylindrical portion of heat sink 360 may have a radius of approximately 35 mm to 45 mm. In some aspects, air inlet 310 may be a plurality of apertures arranged around the perimeter of sidewall 306. In some aspects, side wall 306 can have between 4 to 9 apertures forming air inlet 310.

In some aspects, therapy module 30 has an attachment surface 380 to be coupled with attachment seat 240 of main device 20. In some aspects, attachment surface 380 is bottom surface of bottom cover 304. In some aspects, attachment surface 380 can include a center protrusion 386 and a securement recess 382 and a securement protrusion 383 outside of and along a perimeter of center protrusion 386. In some aspects, securement recess 382 includes electrical contacts 384. In some aspects, electrical contacts 384 can be female electrical contacts that are configured to receive electrical contacts 244 of securement protrusion 242 in main device 20. In some aspects, securement protrusion 383 is configured for mechanically coupling with securement recess 243 in the head portion 220 of main device 20. In additional or alternative aspects, securement protrusion 242 may include male or female electrical contacts for electrically coupling with respective components in main device 20. It will be appreciated that the male and female electrical contacts in securement recess 382 and/or securement protrusion 383 can be reversed. In some aspects, attachment surface 380 includes magnets 390 for magnetically coupling magnets 290 of attachment seat 240. In some aspects, magnets 390 are arranged underneath attachment surface 380 outside of and along the perimeter of center hole 388.

When therapy module 30 is coupled with main device 20, center protrusion 386 is disposed within center recess 246, and securement recess 382 engages with securement protrusion 242, securement protrusion 383 engages with securement recess 243. Further, electrical contacts 384 electrically couples with electrical contacts 244 to transmit micro-current for the micro-current therapy and electric power for the temperature treatment. In some aspects, attachment surface 380 includes magnets 390 for magnetically coupling magnets 290 of attachment seat 240.

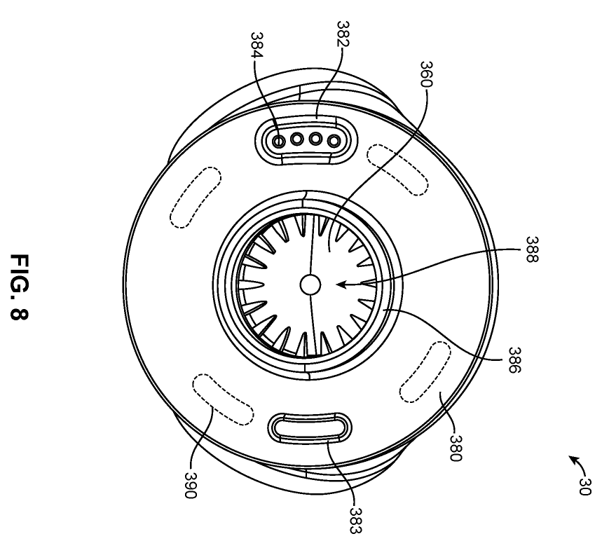

In some aspects, as shown in FIG. 8, center protrusion 386 has a center hole 388 for allowing air flow from therapy module 30 to main device 20 through air pathway 248 defined in center recess 246. In some aspects, center protrusion 386 is a cylindrical protrusion having a diameter that corresponds with the diameter of center recess 246. For example, center protrusion 386 can have a diameter in a range of 15 mm to 25 mm, such as 18 mm to 22 mm, such as 20 mm and a height h in a range of 5 mm to 15 mm, such as 8 mm to 12 mm, such as 10 mm.

In some aspects, system 10 may provide the heating and cooling therapy with different temperature levels. In some aspects, control buttons 212 may include one or more buttons for configuring the temperature settings, such as setting button 215. In some aspects, the heating therapy may implement a range of temperature values including predetermined heating thresholds (e.g., between 30° C and 50° C, or alternatively between 35° C and 43° C) as a first temperature setting. Examples of discrete values within the range for the heating mode may include, for example, values of 35° C, 39° C, and 43° C. In some other aspects, the temperature range for each mode may vary, such that each range may be greater (e.g., between 20° C and 60° C) or smaller (e.g., between 38° C and 40° C).

Similarly, in some aspects, the cooling therapy may implement a range of temperate values including a predetermined cooling threshold (e.g., below 20° C, or alternatively between 8° C and 12° C) as a second temperature setting. Examples of discrete values within the range for the cooling mode may include, for example, values of 12° C, 10° C, and 8° C. In some aspects, the heating and cooling temperature values of system 10 may be specifically chosen to maximize the benefits and safety of the treatment directly on the user’s skin without concern for skin irritation, burns, etc. In some other aspects, the temperature range for each mode may vary, such that each range may be greater (e.g., between 3° C and 30° C) or smaller (e.g., between 10° C and 18° C.).

In some aspects, system 10 may provide the micro-current therapy with different current levels. In some aspects, control buttons 212 may include one or more buttons for configuring the current settings, such as setting button 215. In some aspects, the micro-current therapy may implement a range of microcurrent intensity settings, including predetermined microcurrent thresholds (e.g., between 100 to 500 microamps). Examples of discrete values within the range for the microcurrent treatment mode may include, for example, values of 100 μA, 200 μA, 300 μA, 400 μA, and 500 μA. In some aspects, system 10 may automatically apply the micro-current therapy during the temperature therapy, and specific cooling temperature values and micro-current values may be specifically selected to maximize benefits and safety of treatment and to improve cooling and toning effects on user’s skin. In some aspects, a user may elect, from control buttons 212, to not apply the temperature therapy during the micro-current therapy, and may also elect, from control buttons 212, to apply the temperature therapy without the micro-current therapy. In some aspects, a user may elect, from control buttons 212, heating or cooling during the temperature therapy. For example, in some aspects, system 10 may apply only heating or only cooling during the temperature therapy. In some other aspects, system 10 may apply temperature therapy that alternates between the cooling and heating. The alternating mode may include one or more cycles that alternate between the cooling therapy and the heating therapy for predetermined time periods. For example, a cooling therapy (e.g., 8° C) may be maintained for a certain period (e.g., 1 min) and then switched to a heating mode (e.g., of 43° C) for a certain period (e.g., 1 min). In some aspects, the combination of alternating heating and cooling therapy may be beneficial in helping a user maximize recovery of an area of their body that may be fatigued or sore from activity.

In some aspects, the settings for different therapies may be a preset or user configuration settings, e.g., system 10 may be configured to communicate with a user device such as a mobile phone or computer.

FIG. 11 shows a temperature controlled micro-current system 110 according to some aspects. In some aspects, system 110 includes a main device 120 similar to main device 20 discussed above and a therapy module 130 similar to therapy module 30 discussed above.

However, on main device 120, air outlet 1230 can be disposed on a bottom end of handle portion 1210, and there is no outlet on head portion 1220. Air outlet 1230 can be in fluid communication with air inlet 1310 of therapy module 130 when therapy module 130 is coupled with main device 120. Further, applicators 1320 and 1330 on therapy module 130 can have a spherical shape, instead of an ovoid shape of applicators 320, 330 as discussed above.

Unless the context clearly requires otherwise, throughout the description and the claims, the words "comprise," "comprising," and the like are to be construed in an inclusive sense, as opposed to an exclusive or exhaustive sense; that is to say, in the sense of "including, but not limited to." As used herein, the terms "connected," "coupled," or any variant thereof, means any connection or coupling, either direct or indirect, between two or more elements; the coupling of connection between the elements can be physical, logical, or a combination thereof. Additionally, the words "herein," "above," "below," and words of similar import, when used in this application, shall refer to this application as a whole and not to any particular portions of this application. Where the context permits, words in the above Detailed Description of the Aspects using the singular or plural number may also include the plural or singular number respectively. The word "or" in reference to a list of two or more items, covers all of the following interpretations of the word: any of the items in the list, all of the items in the list, and any combination of the items in the list.

The above-detailed description of aspects of the disclosure is not intended to be exhaustive or to limit the teachings to the precise form disclosed above. While specific aspects of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another aspect, instructions or sub-operations of distinct operations may be implemented in an intermittent and/or alternating manner.

The teachings of the disclosure provided herein can be applied to other systems, not necessarily the system described above. The elements and acts of the various aspects described above can be combined to provide further aspects. Any measurements or dimensions described or used herein are merely exemplary and not a limitation on the present disclosure.

Other measurements or dimensions are within the scope of the disclosure.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further aspects of the disclosure.

These and other changes can be made to the disclosure in light of the above Detailed Description of the Aspects. While the above description describes certain aspects of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific aspects disclosed in the specification unless the above Detailed Description of the Aspects section explicitly defines such terms. Accordingly, the actual scope of the disclosure encompasses not only the disclosed aspects, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. §112, ¶6, other aspects may likewise be embodied as a means-plus-function claim, or in other forms, such as being embodied in a computer-readable medium. (Any claims intended to be treated under 35 U.S.C. §112, ¶6 will include the words "means for").

Accordingly, the applicant reserves the right to add additional claims after filing the application to pursue such additional claim forms for other aspects of the disclosure.

Accordingly, although exemplary aspects of the disclosure have been shown and described, it is to be understood that all the terms used herein are descriptive rather than limiting, and that many changes, modifications, and substitutions may be made by one having ordinary skill in the art without departing from the spirit and scope of the disclosure.

What is claimed is:

1. A treatment device for providing micro-current therapy and temperature therapy, the treatment device comprising:
a main device configured to generate electric current and comprising:
a fan; and
an air outlet; and
a therapy module removably coupled to the main device, the therapy module configured to provide the micro-current therapy and the temperature therapy and comprising:
a pair of applicators configured to be in contact with skin;
a pair of controllable temperature elements configured to provide the temperature therapy; and
an air inlet,
wherein the fan directs air flow from the air inlet to the air outlet when the therapy module is coupled to the main device.

2. The treatment device of claim 1, wherein the pair of applicators comprises an anode electrode and a cathode electrode configured to provide the micro-current therapy while the applicators are in contact with the skin.

3. The treatment device of claim 1, wherein each applicator of the pair of applicators has an ovoid shape comprising a broader base tapering toward a narrower tip along a major axis.

4. The treatment device of claim 3, wherein a distance between the broader bases of the pair of applicators is in a range of 30 mm to 35 mm.

5. The treatment device of claim 3, wherein a distance between the narrower tips of the pair of applicators is in a range of 20 mm to 25 mm.

6. The treatment device of claim 1, wherein the therapy module further comprises a housing comprising an upper cover, a bottom cover, and a sidewall between the upper cover and the bottom cover,
wherein the air inlet is disposed on the sidewall, and wherein the pair of applicators is coupled to the upper cover.

7. The treatment device of claim 1, wherein each one of the pair of applicators has a maximum height as measured from the upper cover in a range of 13 mm to 22 mm.

8. The treatment device of claim 1, wherein the main device comprises a handle portion and a head portion, and wherein the air outlet is disposed on the head portion.

9. The treatment device of claim 8, wherein the main device comprises two air outlets disposed on opposite sides of the head portion.

10. The treatment device of claim 8, wherein an angle between the head portion and a vertical direction of the handle portion is in a range of 30 degrees to 80 degrees.

11. The treatment device of claim 1, wherein the treatment device is configured to administer the micro-current therapy and the temperature therapy simultaneously.

12. The treatment device of claim 11, wherein the micro-current therapy comprises a current in a range of 100 microamps to 500 microamps.

13. The treatment device of claim 11, wherein the temperature therapy is a heating therapy providing a temperature in a range of 30° C and 50° C.

14. The treatment device of claim 13, wherein the temperature therapy is a cooling therapy providing a temperature below 20° C.

15. The treatment device of claim 1, wherein the therapy module further comprises a heat sink to provide heat transfer to the controllable temperature elements.
